(19) United States
(12) Patent Application Publication
(10) Pub. No.: US 2024/0352159 A1
(43) Pub. Date: Oct. 24, 2024

(54) PROCESS FOR PREPARING POLYALPHA-OLEFINS

(71) Applicant: SABIC GLOBAL TECHNOLOGIES B.V., Bergen op Zoom (NL)

(72) Inventors: Nicolaas Hendrika Friederichs, Bengaluru (IN); William GAUTHIER, Houston, TX (US); Paulette HAZIN, Sugar Land, TX (US)

(21) Appl. No.: 18/682,304

(22) PCT Filed: Aug. 10, 2022
(86) PCT No.: PCT/EP2022/072445

§ 371 Date: Feb. 8, 2024

Related U.S. Application Data
(60) Provisional application No. 63/231,734, filed on Aug. 11, 2021.

(51) Int. Cl. C08F 4/76; C08F 110/04; C08F 110/14

(52) U.S. Cl. CPC C08F 4/76; C08F 110/04; C08F 110/14

(57) ABSTRACT

The invention relates to a process for preparing polyalpha-olefins using a catalyst composition comprising a reaction product of an organometallic complex and a co-catalyst, wherein the comprising an organometallic complex is represented by the general formula: LMXn wherein: (i) ‘L’ is an organic ligand; (ii) ‘M’ is a transition metal having a valency of ‘p’, wherein the metal ‘M’ is selected from Ti, Zr, and Hf; (iii) ‘X’ is an anionic ligand to the metal ‘M’, and wherein ‘X’ is selected from the group consisting of halogens, alkyls, aralkyls, alkoxides, amides, and combinations thereof; (iv) ‘n’ is the number of ‘X’ groups and equals p-2.

### CROSS REFERENCE TO RELATED APPLICATIONS

Synthesis of catalysts and ligands disclosed in this application can be found in the following publications incorporated by reference herein: biphenylene bridged metallocenes are described in: WO2015132346, WO2016188999, WO2017118617, WO2021048030, and WO2020043815; Phenylene bridged metallocenes are described in: WO2019145371; Dimethylsilyl bridged metallocenes are described in: WO2018185173, WO2018185170, and WO2018185176. Other ligands are described in: WO2021213836 and EP2970348.

### FIELD OF INVENTION

The invention is directed to a process for preparing polyalpha olefins (PAO) using organometallic based catalyst compositions.

### BACKGROUND

Poly-α-olefins (PAOs; polyalpha-olefins) comprise a class of hydrocarbons manufactured by the catalytic oligomerization of linear alpha-olefins (LAOs). Polyalpha-olefins (PAOs) have certain advantageous properties such as suitable flow properties at low temperatures, relatively high thermal and oxidative stability, which make them suitable for application in lubricant oil. However, polyalpha-olefins often suffer from the drawback of having limited oxidative stability, limited biodegradability and limited additive miscibility thereby impeding its use in high-performance gear oils and fast biodegradable oils. One possible reason for this limitation, is that structurally polyalpha-olefins often include tertiary hydrogen, which is prone to oxidation. Therefore, it would be desirable to minimize the presence of tertiary hydrogen so as to improve oxidation resistance of polyalpha-olefins.

Several catalyst systems have been used for synthesizing polyalpha-olefins with the aim of improving the properties of polyalpha-olefins. For example, polyalpha-olefins are often synthesized by a two-step reaction sequence from linear alpha-olefins, using a boron trifluoride catalyst in conjunction with a protic catalyst such as water, alcohol, or a weak carboxylic acid. However, it has been observed that boron trifluoride catalysts cause excess skeletal branching during the oligomerization process. An increase in the amount of skeletal branching directly correlates with an increase in the number of tertiary hydrogens in the molecule, which are prone to oxidation, and therefore exhibit poor stability when used in lubricants. In another example, polyalpha-olefins may be produced using conventional Friedel-Crafts catalysts. The polyalpha-olefins that are so produced, have excess short chain branches, such as methyl and ethyl short side chains, even though the feed olefins do not contain such short branches. The presence of short chain branches is less desirable for superior lubricant properties, including that of suitable viscosity and volatility properties.

In another example, reduced chromium oxide on silica gel have been reported to be used as a catalyst suitable to polymerize C₆ to C₂₀ alpha-olefins. It is believed that the reduced chromium oxide on silica gel catalyst, polymerizes the lower alpha-olefins, such as 1-butene or 1-hexene, at a higher rate than the alpha-olefins of 1-decene, 1-dodecene or larger alpha-olefins. As a result, the polymers so formed tend to be more blocky or more inhomogeneous and are detrimental to the product viscosity and low temperature properties as required in lubricants.

In yet another example, Ziegler-Natta type catalysts have also been reported to copolymerize mixed alpha-olefin monomers. However, Ziegler-Natta catalysts are generally suitable for synthesizing high molecular weight polymers and not necessarily suitable for synthesizing relatively low molecular weight polyalpha-olefins. Furthermore, according to all literature reports, Ziegler-Natta catalysts usually have higher reactivity towards smaller alpha-olefins, such as propylene, 1-butene, 1-pentene or 1-hexene, than towards higher alpha-olefins, such as 1-decene, 1-dodecene, or larger 1-olefins, leading towards the buildup to higher molecular weight polymers (Macromolecular Chemistry and Physics, 195, 2805 (1994) or Krentsel et al., Polymers and Copolymers of Higher alpha-Olefins, Munchen: Carl Hanser Verlag, 1997. Page 286, FIG. 8.5: illustrating the dependency of reactivity of alpha-olefins on chain length for Ziegler catalysts). This difference in catalyst reactivity results in heterogeneous chemical structures for the copolymers, which are not random copolymers and have high degree of blockiness. Both characteristics are detrimental for lube properties.

In the past, single site catalysts have been used for synthesizing polyalpha-olefins described in patent literature such as U.S. Pat. No. 7,129,197 and US20070225533A1. Although the catalysts systems described in the patents are promising, the properties of the polyalpha-olefins can be further improved upon by modifying the characteristics of an organometallic ligand or designing a new ligand for constituting a single site catalyst. Further, there is a scope of further improving the performance of single site catalyst in terms of comonomer incorporation in a copolymerization reaction where it has been observed that monomer incorporation efficiency rapidly decreases with higher carbon number monomers (McDaniel, M. P. et al, Macromolecules 2010, 43, 8836-8852. FIG. 4). As may be appreciated by a skilled person, a catalyst suitable for polymerization to produce polyolefins, may not be suitable for oligomerization as has been observed for certain Ziegler-Natta catalyst or certain single site catalysts, which when used tend to produce high molecular weight polymers rather than oligomers and in addition may suffer from low productivity due to the low reactivity of the higher alpha-olefin monomers.

Therefore, there is a need for developing a process for producing polyalpha-olefins using a suitable catalyst system that can produce poly-alpha olefins with high oxidative stability, relatively uniform chemical structures, and a suitable viscosity index at appreciable productivity.

### SUMMARY

Accordingly, the need for developing a process for producing polyalpha-olefins using suitable catalyst, is addressed by a process as described in this present disclosure. In various embodiments of the invention, the invention is directed to a process for preparing polyalpha-olefins, comprising the steps of:

a. providing a feed stream comprising one or more alpha-olefin monomer, wherein the alpha-olefin monomer comprises four to thirty (4-30) carbon atoms;
b. contacting the feed stream with a catalyst composition comprising a reaction product of an organometallic complex and a co-catalyst, wherein the organometallic complex is represented by the general formula: LMXₙ wherein:
I. ‘L’ is an organic ligand;
II. ‘M’ is a transition metal having a valency of ‘p’, wherein the metal ‘M’ is selected from Ti, Zr, and Hf;
III. ‘X’ is an anionic ligand to the metal ‘M’, and wherein ‘X’ is selected from the group consisting of halogens, alkyls, aralkyls, alkoxides, amides, and combinations thereof;
IV. ‘n’ is the number of ‘X’ groups and equals p-2;
   wherein the organic ligand is selected from the group consisting of:
i. a bridging group bonded to two hydrocarbyl groups, each comprising a substituted or an unsubstituted cyclopentadienyl group; wherein the bridging group contains at least one sp² hybridized carbon atom bonded to at least one of the hydrocarbyl groups;
ii. a substituted or an unsubstituted 2,2′-biphenylene-bridging group, wherein the bridging group is bonded to two hydrocarbyl groups, each comprising a substituted or an unsubstituted cyclopentadienyl group;
iii. a disubstituted alkyl-silyl bridging group, wherein the bridging group is bonded to two substituted or unsubstituted indenyl groups, wherein at least one substituted or an unsubstituted indenyl group is bonded to the bridging group at the second position of the indenyl group;
iv. a disubstituted alkyl-silyl bridging group, wherein the bridging group is bonded to a tetra-alkyl substituted cyclopentadienyl group and to a 1-indenyl group, wherein the 1-indenyl group comprises a substituent at the second position, wherein the substituent is selected from C₃-C₂₀ alkyl group or C₆-C₂₀ aryl group;
v. a substituted or an unsubstituted 1, 2-phenylene bridging group bonded to a substituted or an unsubstituted 1-indenyl group and to a substituted or an unsubstituted 2-indenyl group;
vi. a substituted or an unsubstituted 2,2′-biphenylene-bridging group bonded to two hydrocarbyl groups, each hydrocarbyl group comprising a substituted indenyl group, wherein the bridging group is bonded at the second position of each of the substituted indenyl group;
vii. a bridging group comprising a substituted or an unsubstituted styryl moiety bonded to a substituent selected from a Group 16 element of the IUPAC Periodic Table (e.g., oxygen) and to a substituted cyclopentadienyl group, wherein the bridging group is bonded to a substituted cyclopentadienyl group at the alpha position of the styryl moiety;
viii. a substituted or an unsubstituted 2,2′-biphenylene bridging group bonded to a substituted or an unsubstituted indenyl group and to a substituent selected from a Group 15 or a Group 16 element of the IUPAC Periodic Table (e.g., nitrogen or oxygen);
ix. a substituted or an unsubstituted 2,2′-biphenylene group bonded to a substituent (A) and to a substituent (B), wherein substituent (A) is selected from a Group 15 element of the IUPAC Periodic Table (e.g., nitrogen) and substituent (B) is selected from a Group 15 or a Group 16 element of the IUPAC Periodic Table (e.g., oxygen), further wherein the group 15 element of substituent (A) is covalently bonded to two sp² hybridized carbon atoms;
x. a bridging group comprising at least one sp² hybridized carbon atom bonded to two substituted or unsubstituted 2-indenyl groups;
xi. a substituted or an unsubstituted 1,8-naphthalene-bridging group bonded to two substituted or unsubstituted 2-indenyl groups; and
xii. any combination thereof.
   c. oligomerizing the one or more alpha-olefin monomer in presence of the catalyst composition under conditions sufficient to produce polyalpha-olefins.

In another aspect of the invention, use of any of the organometallic complexes set forth in the present disclosure as a catalyst in a process for producing polyalpha-olefins is provided.

The present disclosure includes, without limitation, the following embodiments.

Embodiment 1: A process for preparing polyalpha-olefins, comprising:
a. providing a feedstream comprising one or more alpha-olefin monomer, wherein the alpha-olefin monomer comprises four to thirty (4-30) carbon atoms;
b. contacting the feedstream with a catalyst composition comprising a reaction product of an organometallic complex and a co-catalyst, wherein the organometallic complex is represented by the general formula:

LMXₙ
wherein:
I. ‘L’ is an organic ligand;
II. ‘M’ is a transition metal having a valency of ‘p’, wherein the metal ‘M’ is selected from Ti, Zr, and Hf;
III. ‘X’ is an anionic ligand to the metal ‘M’, and wherein ‘X’ is selected from the group consisting of halogens, alkyls, aralkyls, alkoxides, amides, and combinations thereof;
IV. ‘n’ is the number of ‘X’ groups and equals p-2;
   wherein the organic ligand ‘L’ is selected from the group consisting of:
i. a bridging group bonded to two hydrocarbyl groups, each comprising a substituted or an unsubstituted cyclopentadienyl group; wherein the bridging group contains at least one sp² hybridized carbon atom bonded to at least one of the hydrocarbyl groups;
ii. a substituted or an unsubstituted 2,2′-biphenylene-bridging group, wherein the bridging group is bonded to two hydrocarbyl groups, each comprising a substituted or an unsubstituted cyclopentadienyl group;
iii. a di-substituted alkyl-silyl bridging group, wherein the bridging group is bonded to two substituted or unsubstituted indenyl groups, wherein at least one substituted or unsubstituted indenyl group is bonded to the bridging group at the second position of the indenyl group;
iv. a di-substituted alkyl-silyl bridging group, wherein the bridging group is bonded to a tetra-alkyl substituted cyclopentadienyl group and to a 1-indenyl group, wherein the 1-indenyl group comprises a substituent at the second position wherein the substituent is selected from C₃-C₂₀ alkyl or C₆-C₂₀ aryl group;
v. a substituted or an unsubstituted 1, 2-phenylene bridging group bonded to a substituted or an unsubstituted 1-indenyl group and to a substituted or an unsubstituted 2-indenyl group;
vi. a substituted or an unsubstituted 2,2′-biphenylene-bridging group bonded to two hydrocarbyl groups, each hydrocarbyl group comprising a substituted indenyl group, wherein the bridging group is bonded at the second position of each of the substituted indenyl group;
vii. a bridging group comprising a substituted or an unsubstituted styryl moiety bonded to a substituent selected from a Group 16 element of the IUPAC Periodic Table and to a substituted cyclopentadienyl group, wherein the bridging group is bonded to a substituted cyclopentadienyl group at the alpha position of the styryl moiety;
viii. a substituted or an unsubstituted 2,2′-biphenylene bridging group bonded to a substituted or an unsubstituted indenyl group and to a substituent selected from a Group 15 or a Group 16 element of the IUPAC Periodic Table;
ix. a substituted or an unsubstituted 2,2′-biphenylene group bonded to a substituent (A) and to a substituent (B), wherein substituent (A) is selected from a Group 15 element of the IUPAC Periodic Table and substituent (B) is selected from a Group 15 or a Group 16 element of the IUPAC Periodic Table, further wherein the group 15 element of substituent (A) is covalently bonded to two sp² hybridized carbon atoms;
x. a bridging group comprising at least one sp² hybridized carbon atom bonded to two substituted or unsubstituted 2-indenyl groups;
xi. a substituted or an unsubstituted 1,8-naphthalene-bridging group bonded to two substituted or unsubstituted 2-indenyl groups; and
xii. any combination thereof
   c. oligomerizing the one or more alpha-olefin monomer in presence of the catalyst composition under conditions sufficient to produce polyalpha-olefins.

Embodiment 2: The process of Embodiment 1, wherein the organic ligand ‘L’ comprises a substituted or an unsubstituted 2,2′-biphenylene-bridging group, wherein the bridging group is bonded to two hydrocarbyl groups, each comprising a substituted or an unsubstituted cyclopentadienyl group, such as wherein the organometallic complex is selected from the group consisting of:

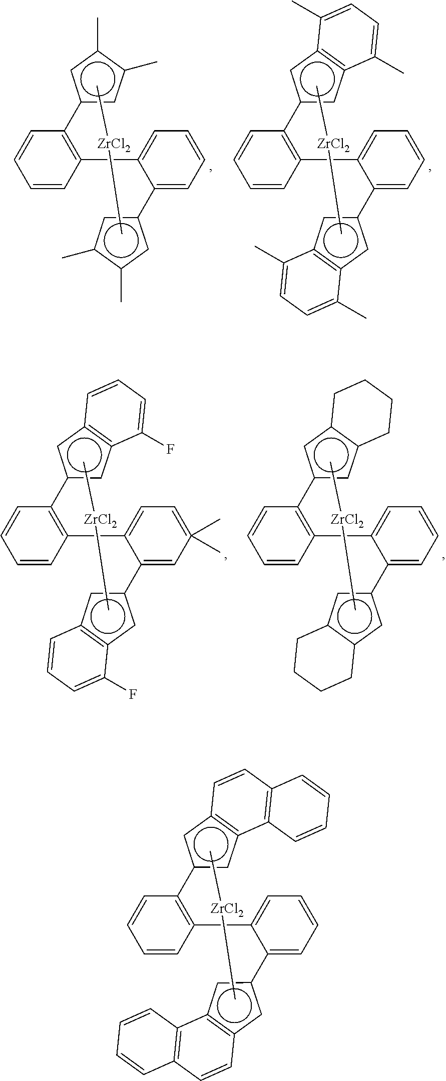

and any combinations thereof.

Embodiment 3: The process of Embodiment 1 or 2, wherein the organometallic complex is:

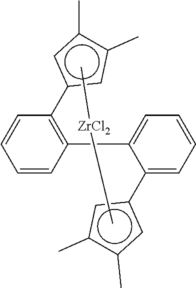

Embodiment 4: The process of Embodiment 1 or 2, wherein the organometallic complex is

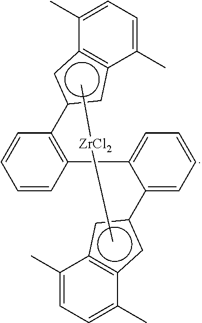

Embodiment 5: The process of Embodiment 1 or 2, wherein the organometallic complex is:

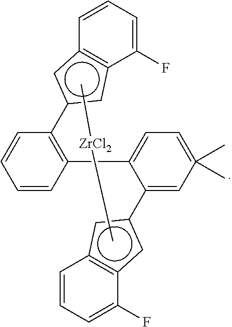

Embodiment 6: The process of Embodiment 1 or 2, wherein the organometallic complex is:

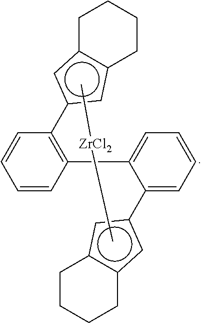

Embodiment 7: The process of Embodiment 1 or 2, wherein the organometallic complex is:

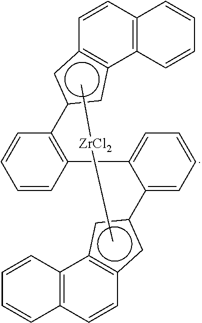

Embodiment 8: The process of Embodiment 1, wherein the organic ligand ‘L’ comprises a di-substituted alkyl-silyl bridging group, wherein the bridging group is bonded to two substituted or unsubstituted indenyl groups, wherein at least one substituted or an unsubstituted indenyl group is bonded to the bridging group at the second position of the indenyl group, such as wherein the organometallic complex is selected from the group consisting of:

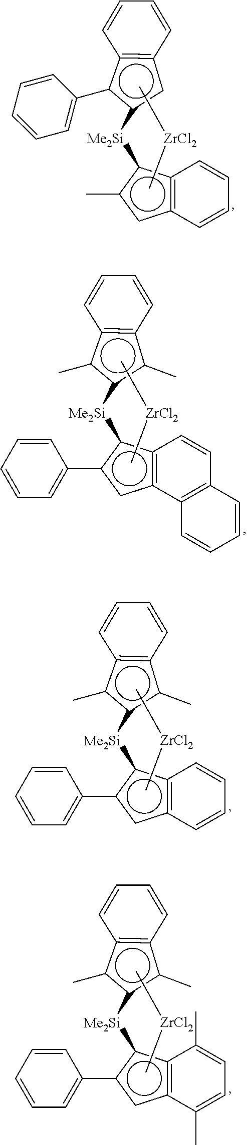

and any combinations thereof.

Embodiment 9: The process of Embodiment 1 or Embodiment 8, wherein the organometallic complex is:

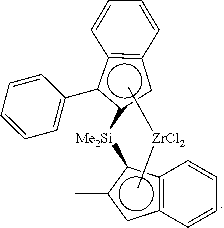

Embodiment 10: The process of Embodiment 1 or Embodiment 8, wherein the organometallic complex is:

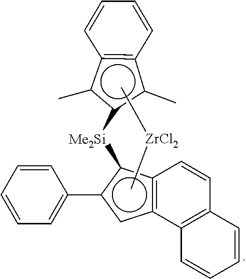

Embodiment 11: The process of Embodiment 1 or Embodiment 8, wherein the organometallic complex is:

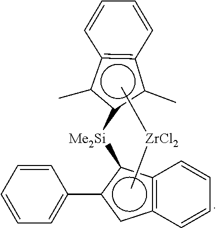

Embodiment 12: The process of Embodiment 1 or Embodiment 8, wherein the organometallic complex is:

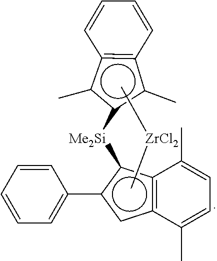

Embodiment 13: The process of Embodiment 1, wherein the organic ligand ‘L’ comprises a di-substituted alkyl-silyl bridging group, wherein the bridging group is bonded to a tetra-alkyl substituted cyclopentadienyl group and to a 1-indenyl group, wherein the 1-indenyl group comprises a substituent at the second position wherein the substituent is selected from C₃-C₂₀ alkyl or C₆-C₂₀ aryl group, such as wherein the organometallic complex is selected from the group consisting of:

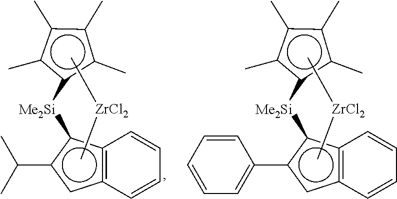

and combinations thereof.

Embodiment 14: The process of Embodiment 1 or Embodiment 13, wherein the organometallic complex is:

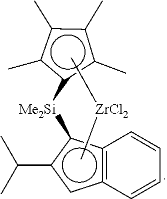

Embodiment 15: The process of Embodiment 1 or Embodiment 13, wherein the organometallic complex is:

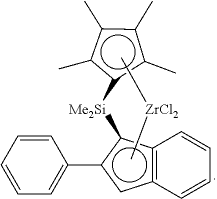

Embodiment 16: The process of Embodiment 1, wherein the organic ligand ‘L’ comprises a substituted or an unsubstituted 1, 2-phenylene bridging group bonded to a substituted or an unsubstituted 1-indenyl group and to a substituted or an unsubstituted 2-indenyl group; such as wherein the organometallic complex is:

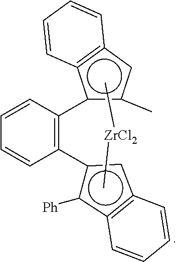

Embodiment 17: The process of Embodiment 1, wherein the organic ligand ‘L’ comprises a substituted or an unsubstituted 2,2′-biphenylene-bridging group bonded to two hydrocarbyl groups, each hydrocarbyl group comprising a substituted indenyl group, wherein the bridging group is bonded at the second position of each of the substituted indenyl group, such as wherein the organometallic complex is:

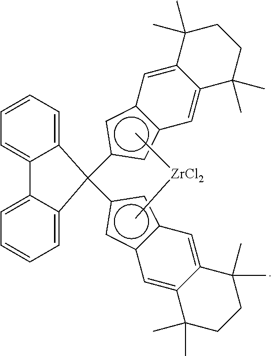

Embodiment 18: The process of Embodiment 1, wherein the organic ligand ‘L’ comprises a bridging group comprising a substituted or an unsubstituted styryl moiety bonded to a substituent selected from a Group 16 element of the IUPAC Periodic Table and to a substituted cyclopentadienyl group, wherein the bridging group is bonded to a substituted cyclopentadienyl group at the alpha position of the styryl moiety; such as wherein the organometallic complex is selected from the group consisting of:

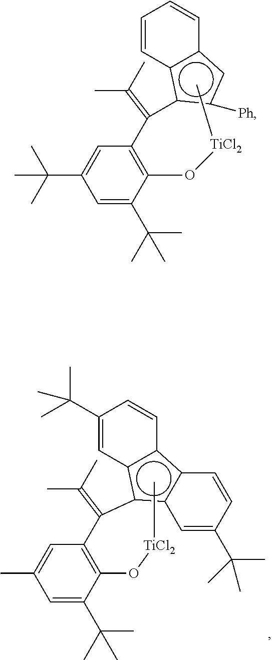

and any combinations thereof.

Embodiment 19: The process of Embodiment 1 or Embodiment 18, wherein the organometallic complex is:

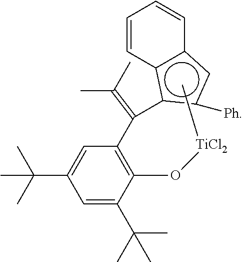

Embodiment 20: The process of Embodiment 1 or Embodiment 18, wherein the organometallic complex is:

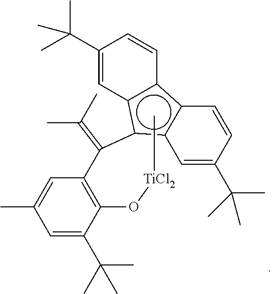

Embodiment 21: The process of Embodiment 1, wherein the organic ligand ‘L’ comprises a substituted or an unsubstituted 2,2′-biphenylene bridging group bonded to a substituted or an unsubstituted indenyl group and to a substituent selected from a Group 15 or a Group 16 element of the IUPAC Periodic Table, such as wherein the organometallic complex is:

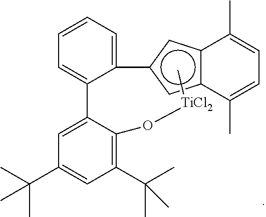

Embodiment 22: The process of Embodiment 1, wherein the organic ligand ‘L’ comprises a substituted or an unsubstituted 2,2′-biphenylene group bonded to a substituent (A) and to a substituent (B), wherein substituent (A) is selected from a Group 15 element of the IUPAC Periodic Table and substituent (B) is selected from a Group 15 or a Group 16 element of the IUPAC Periodic Table, further wherein the group 15 element of substituent (A) is covalently bonded to two sp² hybridized carbon atoms, such as wherein the organometallic complex is:

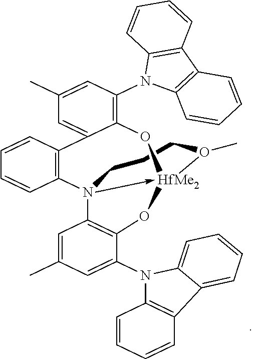

Embodiment 23: The process of Embodiment 1, wherein the organic ligand ‘L’ comprises a bridging group comprising at least one sp² hybridized carbon atom bonded to two substituted or unsubstituted 2-indenyl groups, such as wherein the organometallic complex is selected from the group consisting of:

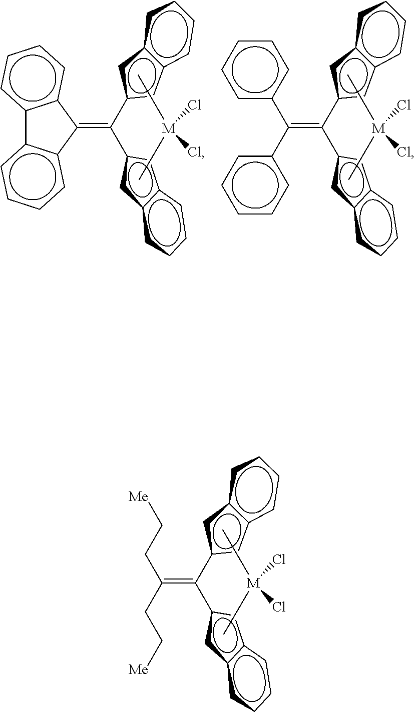

and combinations thereof, wherein ‘M’ is a transition metal having a valency of 4′, wherein the metal ‘M’ is selected from Ti, Zr, and Hf.

Embodiment 24: The process of Embodiment 1 or Embodiment 23, wherein the organometallic complex is:

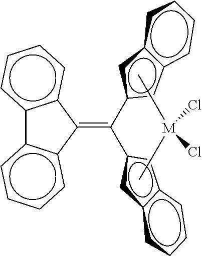

wherein ‘M’ is a transition metal having a valency of ‘4’, wherein the metal ‘M’ is selected from Ti, Zr, and Hf.

Embodiment 25: The process of Embodiment 1 or Embodiment 23, wherein the organometallic complex is:

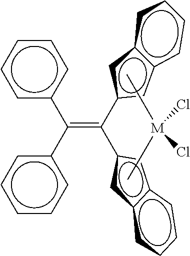

wherein ‘M’ is a transition metal having a valency of ‘4’, wherein the metal ‘M’ is selected from Ti, Zr, and Hf.

Embodiment 26: The process of Embodiment 1 or Embodiment 23, wherein the organometallic complex is:

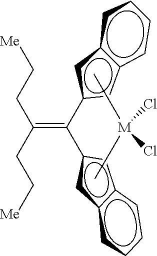

wherein ‘M’ is a transition metal having a valency of ‘4’, wherein the metal ‘M’ is selected from Ti, Zr, and Hf.

Embodiment 27: The process of Embodiment 1, wherein the organic ligand ‘L’ comprises a substituted or an unsubstituted 1,8-naphthalene-bridging group bonded to two substituted or unsubstituted 2-indenyl groups, such as wherein the organometallic complex is:

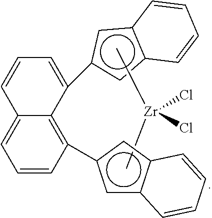

Embodiment 28: The process of any one of Embodiments 1 to 27, wherein the one or more alpha-olefin monomer present in the feedstream is selected from the group consisting of 1-butene, 1-pentene, 1-hexene, 1-heptene, 1-octene, 1-nonene, 1-decene, 1-undecene, 1-dodecene, 1-tridecene, 1-tetradecene, 1-hexadecene, 1-octadecene, 1-eicosene, 4-methyl-1-pentene, 5-methyl-1-nonene, 3-methyl-1-pentene, 3,5,5-trimethyl-1-hexene, vinylcyclohexene, and any combinations thereof.

Embodiment 29: The process of any one of Embodiments 1 to 28, wherein the one or more alpha-olefin monomer is oligomerized at a reactor temperature ranging between 25° C. to 150° C.

Embodiment 30: The process of any one of Embodiments 1 to 29, wherein the one or more alpha-olefin monomer is oligomerized at a reactor pressure ranging between atmospheric pressure to about 50 psia.

Embodiment 31: The process of any one of Embodiments 1 to 30, wherein the poly-alpha olefins once formed may be hydrogenated in presence of a hydrogenation catalyst to form a partially saturated or a completely saturated polyalpha-olefin oligomers.

Embodiment 32: The process of any one of Embodiments 1 to 31, wherein the cocatalyst is selected from an aluminium containing cocatalyst, a boron-containing cocatalyst, zinc containing cocatalyst, or a combination thereof.

Embodiment 33: Use of the any of the organometallic complexes set forth in any one of Embodiments 1 to 32 as a catalyst in a process for producing polyalpha-olefins.

Other objects, features and advantages of the invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from various specific embodiments may be combined with features from any of the other embodiments. In further embodiments, additional features may be added to the specific embodiments described herein.

### DETAILED DESCRIPTION

The invention is based, in part, on the discovery of a process for preparing polyalpha-olefins using a catalyst composition comprising an organometallic complex. Advantageously, the polyalpha-olefins prepared from the process of the present invention, demonstrates a balance of high oxidative stability, relatively uniform chemical structure, and a suitable viscosity index at appreciable productivity.

The following paragraph includes definitions of various terms, expressions and phrases used throughout this specification.

The use of the words “a” or “an” when used in conjunction with the term “comprising,” “including,” “containing,” or “having” in the claims or the specification may mean “one,” but it is also consistent with the meaning of “one or more,” “at least one,” and “one or more than one.” The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. The method of the invention can “comprise,” “consist essentially of,” or “consist of” particular ingredients, components, compositions, etc., disclosed throughout the specification.

The term “polyalpha-olefin” as used herein refers to hydrocarbons or oligomers manufactured by the oligomerization of alpha-olefin monomers. Polyalpha-olefins include for example C₂₀₋₂₄ dimers, C₃₀₋₃₆ trimers, C₄₀₋₄₈ tetramers, C₅₀₋₆₀ pentamers, and C₆₀₊ heavies. Generally, suitable alpha-olefins are represented by the following formula: CH₂═CHR where R can be any hydrocarbyl group, such as alkyl, aryl, or aralkyl. For the avoidance of doubt, polyalpha-olefins are distinct from polyolefins (e.g. polyethylene, polypropylene) where polyalpha-olefins are oligomers having relatively a much lower molecular weight, with a much lower number of repeating monomeric units compared to polyolefins having high molecular weight and large number of repeating monomeric units. In various embodiments of the invention, the polyalpha-olefins obtained from the process of the present invention has an average molecular weight (Mw) expressed in terms of g/mol of 200 to 32000, alternatively 300 to 25,000, alternatively 400 to 18,000. The molecular weight provided for the polyalpha-olefins is lower than that of the polyolefins such as polyethylene, which typically have molecular weight in the hundreds of thousands.

As a general matter, any carbon atom of the various organometallic complexes of the present disclosure may be substituted or unsubstituted, meaning the carbon atom can be attached to one or more hydrogen atoms or one or more of the hydrogen atoms may be replaced with a different moiety, with example substituents including a C₁-C₃₀ alkyl group, a C₆-C₃₀ aryl group, or a heteroatom atom.

The expression “substituted or an unsubstituted cyclopentadienyl group” means that a cyclopentadienyl ring may be substituted by one or more substituents selected from hydrogen, a C₁-C₃₀ alkyl group, a C₆-C₃₀ aryl group, a heteroatom atom, or alternatively that a cyclopentadienyl ring is fused to one or more ring system to form a cyclopentadienyl fused ring system. Non-limiting examples of cyclopentadienyl fused ring system includes an indenyl ring, a fused indenyl ring, a substituted indenyl ring, a tetra-hydro indene, a fluorenyl ring, a substituted fluorenyl ring. The fused indenyl ring comprises for example at least one, alternatively two, alternatively three ring systems fused to an indenyl ring. Alternatively, a cyclopentadienyl fused ring system may include one or more ring systems fused with a cyclopentadienyl ring that may be completely saturated or partially saturated in order to attenuate catalyst performance.

The expression “substituted or an unsubstituted indenyl group” means that an indenyl ring may be substituted by one or more substituents selected from hydrogen, a C₁-C₃₀ alkyl group, a C₆-C₃₀ aryl group, a heteroatom atom, or alternatively the indenyl ring is fused to one or more ring system to form an indenyl fused ring system.

The expression “substituted or an unsubstituted 2,2′-biphenylene group” means that a 2,2′-biphenylene group may be substituted by one or more substituents selected from hydrogen, a C₁-C₃₀ alkyl group, a C₆-C₃₀ aryl group, or a heteroatom atom.

The expression “substituted or an unsubstituted 1, 2-phenylene bridging group” means that a 1, 2-phenylene bridging group may be substituted by one or more substituents selected from hydrogen, a C₁-C₃₀ alkyl group, a C₆-C₃₀ aryl group, or a heteroatom atom.

The expression “substituted or an unsubstituted styryl moiety” means that a styryl moiety may be substituted by one or more substituents selected from hydrogen, a C₁-C₃₀ alkyl group, a C₆-C₃₀ aryl group, or a heteroatom atom.

The expression “substituted or an unsubstituted 1,8-naphthalene-bridging group” means that a 1,8-naphthalene-bridging group may be substituted by one or more substituents selected from hydrogen, a C₁-C₃₀ alkyl group, a C₆-C₃₀ aryl group, or a heteroatom atom.

The expression “substituted indenyl group” or “substituted 1-indenyl group” or “substituted 2-indenyl group” means that an indenyl ring may be substituted by one or more substituents selected from hydrogen, a C₁-C₃₀ alkyl group, a C₆-C₃₀ aryl group, a heteroatom, or alternatively the indenyl ring is fused to one or more ring system to form a fused indenyl ring system.

The expression “sp² hybridized carbon atoms” as used herein includes a carbon atom of an aromatic ring, for example, a carbon atom of a phenyl ring and also includes a carbon atom of an alkenyl group, wherein such carbon atom has sp² hybridization.

The expression “tetra-alkyl substituted cyclopentadienyl group” means a cyclopentadienyl group having a cyclopentadienyl ring substituted by four C₁-C₃₀ alkyl groups.

The term “hydrocarbyl” refers to an organic radical primarily composed of carbon and hydrogen, which may be aliphatic, alicyclic, aromatic, a hydrocarbon ring system, or a fused cyclic ring system, for example a cyclopentadienyl ring or an indenyl ring, or combinations thereof, e.g., aralkyl or alkaryl.

The expression “Group 15 element of the IUPAC Periodic Table” means any of the following elements: N, P, As, Sb, Bi.

The expression “Group 16 element of the IUPAC Periodic Table” means any of the following elements: O, S, Se, Te, Po.

Accordingly, the invention is directed to a process for producing polyalpha-olefins, comprising the steps of:

a. providing a feed stream comprising one or more alpha-olefin monomer, wherein the alpha-olefin monomer comprises four to thirty (4-30) carbon atoms;
b. contacting the feed stream with a catalyst composition comprising a reaction product of an organometallic complex and a co-catalyst, wherein the organometallic complex is represented by the general formula: LMXₙ wherein:
I. ‘L’ is an organic ligand;
II. ‘M’ is a transition metal having a valency of ‘p’, wherein the metal ‘M’ is selected from Ti, Zr, and Hf;
III. ‘X’ is an anionic ligand to the metal ‘M’, and wherein ‘X’ is selected from the group consisting of halogens, alkyls, aralkyls, alkoxides, amides, and combinations thereof;
IV. ‘n’ is the number of ‘X’ groups and equals p-2;
   wherein the organic ligand ‘L’ is selected from the group consisting of:
i. a bridging group bonded to two hydrocarbyl groups, each comprising a substituted or an unsubstituted cyclopentadienyl group; wherein the bridging group contains at least one sp² hybridized carbon atom bonded to at least one of the hydrocarbyl groups;
ii. a substituted or an unsubstituted 2,2′-biphenylene-bridging group, wherein the bridging group is bonded to two hydrocarbyl groups, each comprising a substituted or an unsubstituted cyclopentadienyl group;
iii. a disubstituted alkyl-silyl bridging group, wherein the bridging group is bonded to two substituted or unsubstituted indenyl groups, wherein at least one substituted or an unsubstituted indenyl group is bonded to the bridging group at the second position of the indenyl group;
iv. a disubstituted alkyl-silyl bridging group, wherein the bridging group is bonded to a tetra-alkyl substituted cyclopentadienyl group and to a 1-indenyl group, wherein the 1-indenyl group comprises a substituent at the second position, wherein the substituent is selected from C₃-C₂₀ alkyl or C₆-C₂₀ aryl group;
v. a substituted or an unsubstituted 1, 2-phenylene bridging group bonded to a substituted or an unsubstituted 1-indenyl group and to a substituted or an unsubstituted 2-indenyl group;
vi. a substituted or an unsubstituted 2,2′-biphenylene-bridging group bonded to two hydrocarbyl groups, each hydrocarbyl group comprising a substituted indenyl group, wherein the bridging group is bonded at the second position of each of the substituted indenyl group;
vii. a bridging group comprising a substituted or an unsubstituted styryl moiety bonded to a substituent selected from a Group 16 element of the IUPAC Periodic Table and to a substituted cyclopentadienyl group, wherein the bridging group is bonded to a substituted cyclopentadienyl group at the alpha position of the styryl moiety;
viii. a substituted or an unsubstituted 2,2′-biphenylene bridging group bonded to a substituted or an unsubstituted indenyl group and to a substituent selected from a Group 15 or a Group 16 element of the IUPAC Periodic Table;
ix. a substituted or an unsubstituted 2,2′-biphenylene group bonded to a substituent (A) and to a substituent (B), wherein substituent (A) is selected from a Group 15 element of the IUPAC Periodic Table and substituent (B) is selected from a Group 15 or a Group 16 element of the IUPAC Periodic Table, further wherein the group 15 element of substituent (A) is covalently bonded to two sp² hybridized carbon atoms;
x. a bridging group comprising at least one sp² hybridized carbon atom bonded to two substituted or unsubstituted 2-indenyl group;
xi. a substituted or an unsubstituted 1,8-naphthalene-bridging group bonded to two substituted or an unsubstituted 2-indenyl group; and
xii. any combination thereof.
   c. oligomerizing the one or more alpha-olefin monomer in presence of the catalyst composition under conditions sufficient to produce polyalpha-olefins.

In various embodiments of the invention, the poly-alpha olefins once formed may be hydrogenated in presence of a hydrogenation catalyst to form a partially saturated or a completely saturated polyalpha-olefin oligomers. In various embodiments of the invention, the polyalpha-olefin is hydrogenated by reaction with hydrogen gas in the presence of a catalytic amount (0.1 to 5 wt. %) of a hydrogenation catalyst. Examples of suitable hydrogenation catalysts are metals of Group VIII of the Periodic Table such as iron, cobalt, nickel, rhodium, palladium and platinum. These catalysts may be deposited on alumina, on silica gel, or on activated carbon in preferred embodiments of the invention. Of these catalysts, palladium and nickel are preferred. Palladium on activated carbon and nickel on kieselguhr are especially preferred. In an embodiment of the invention, the synthesized polyalpha-olefins has some degree of unsaturation. The unsaturation is primarily in the form of vinylidene groups. In an aspect of the invention, the oligomer is synthesized as an unsaturated oligomer, and it is subsequently hydrogenated to produce a saturated oligomer.

In various embodiments of the invention, the polyalpha-olefins may have some level of unsaturation, such as the presence of double bonds. The unsaturated bonds may be hydrogenated in a hydrogenation reaction. The hydrogenation reaction can be carried out in the presence or absence of solvents. Solvents are necessary only to increase the volume. Examples of suitable solvents are hydrocarbons such as pentane, hexane, heptane, octane, decane, cyclohexane, methycyclohexane and cyclooctane aromatic hydrocarbons such as toluene, xylene or benzene. The temperature of the hydrogenation reaction may range, for example, from about 150° C. to about 500° C., preferably from about 250° C. to about 350° C. The hydrogenation reaction pressure may be, for example, in the range of 250-1000 psig hydrogen. The hydrogenated oligomeric polyalpha-olefins product is then recovered by conventional procedures. In the hydrogenated product, the double bonds formed in the oligomerization step have been hydrogenated so that the oligomer is a separate type of product. The hydrogenated oligomer may be used in the same manner as the unhydrogenated oligomer.

In various embodiments of the invention, the organic ligand ‘L’ comprises a substituted or an unsubstituted 2,2′-biphenylene-bridging group, wherein the bridging group is bonded to two hydrocarbyl groups, each comprising a substituted or an unsubstituted cyclopentadienyl group, such as wherein the organometallic complex is selected from the group consisting of:

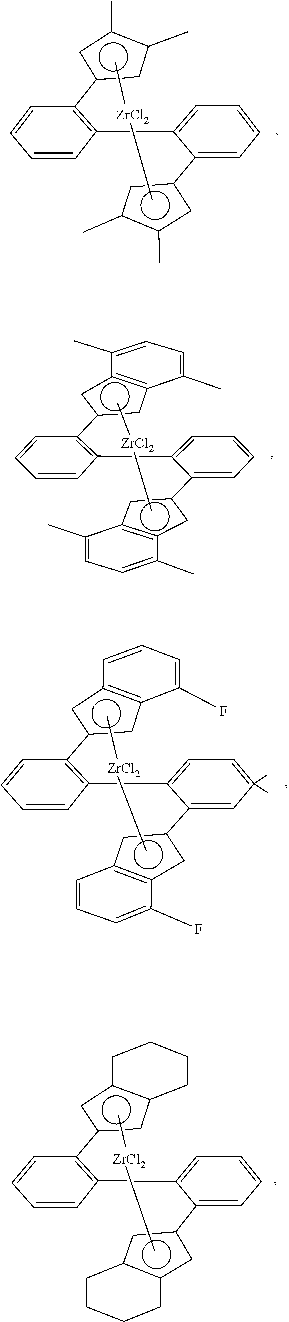

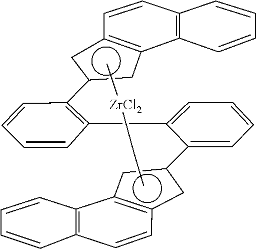

and any combinations thereof.

In various embodiments of the invention, the organic ligand ‘L’ comprises a substituted or an unsubstituted 2,2′-biphenylene-bridging group, wherein the bridging group is bonded to two hydrocarbyl groups, each comprising a substituted or an unsubstituted cyclopentadienyl group, such as wherein the organometallic complex is:

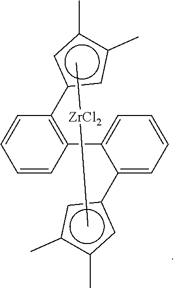

In various embodiments of the invention, the organic ligand ‘L’ comprises a substituted or an unsubstituted 2,2′-biphenylene-bridging group, wherein the bridging group is bonded to two hydrocarbyl groups, each comprising a substituted or an unsubstituted cyclopentadienyl group, such as wherein the organometallic complex is:

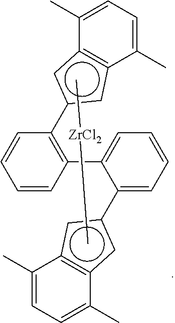

In various embodiments of the invention, the organic ligand ‘L’ comprises a substituted or an unsubstituted 2,2′-biphenylene-bridging group, wherein the bridging group is bonded to two hydrocarbyl groups, each comprising a substituted or an unsubstituted cyclopentadienyl group, such as wherein the organometallic complex is:

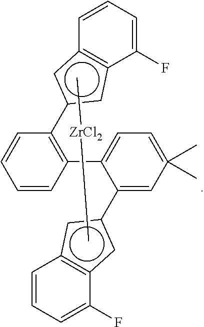

In various embodiments of the invention, the organic ligand ‘L’ comprises a substituted or an unsubstituted 2,2′-biphenylene-bridging group, wherein the bridging group is bonded to two hydrocarbyl groups, each comprising a substituted or an unsubstituted cyclopentadienyl group, such as wherein the organometallic complex is:

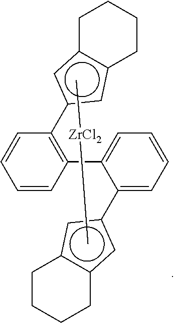

In various embodiments of the invention, the organic ligand ‘L’ comprises a substituted or an unsubstituted 2,2′-biphenylene-bridging group, wherein the bridging group is bonded to two hydrocarbyl groups, each comprising a substituted or an unsubstituted cyclopentadienyl group, such as wherein the organometallic complex is:

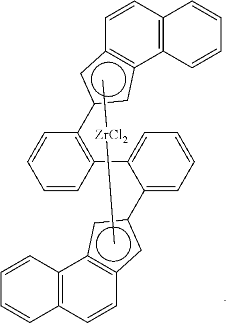

In various embodiments of the invention, the organic ligand ‘L’ comprises a substituted or an unsubstituted 2,2′-biphenylene-bridging group bonded to two hydrocarbyl groups, each hydrocarbyl group comprising a substituted indenyl group, wherein the bridging group is bonded at the second position of each of the substituted indenyl group, such as wherein the organometallic complex is:

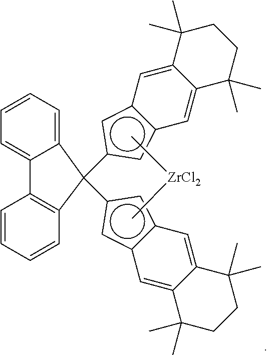

Further examples of organometallic complexes comprising a substituted or an unsubstituted 2,2′-biphenylene-bridging group and methods of making such organometallic complexes can be found, for example, in U.S. Pat. No. 9,938,360 to Vadake Kulangara et al.; U.S. Pat. No. 10,400,048 to Vadake Kulangara et al.; U.S. Pat. No. 11,040,995 to Kulangara et al.; and PCT application W02020/043815A1 to Hendriksen et al., each of which is incorporated by reference in its entirety. See also the synthesis methods disclosed in ‘Synthesis, structure, and properties of chiral titanium and zirconium complexes bearing biaryl strapped substituted cyclopentadienyl ligands’, W. W. Ellis et al, Organometallics 1993, 12, 4391-4401, and ‘Biphenyl-bridged metallocene complexes of titanium, zirconium, and vanadium: syntheses, crystal structures and enantioseparation’, M. E. Huttenloch et al., J. of Organometallic Chemistry 541 (1997), 219-232.

In various embodiments of the invention, the organic ligand ‘L’ comprises a di-substituted alkyl-silyl bridging group, wherein the bridging group is bonded to two substituted or unsubstituted indenyl groups, wherein at least one substituted or an unsubstituted indenyl group is bonded to the bridging group at the second position of the indenyl group, such as wherein the organometallic complex is selected from the group consisting of:

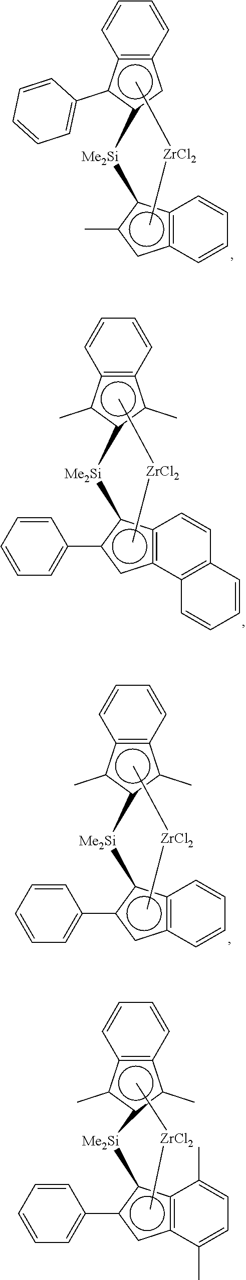

and any combinations thereof.

In various embodiments of the invention, the organic ligand ‘L’ comprises a di-substituted alkyl-silyl bridging group, wherein the bridging group is bonded to two substituted or unsubstituted indenyl groups, wherein one substituted or an unsubstituted indenyl group is bonded to the bridging group at the second position of the indenyl group and one substituted or an unsubstituted indenyl group is bonded to the bridging group at the first position of the indenyl group, such as wherein the organometallic complex is selected from the group consisting of:

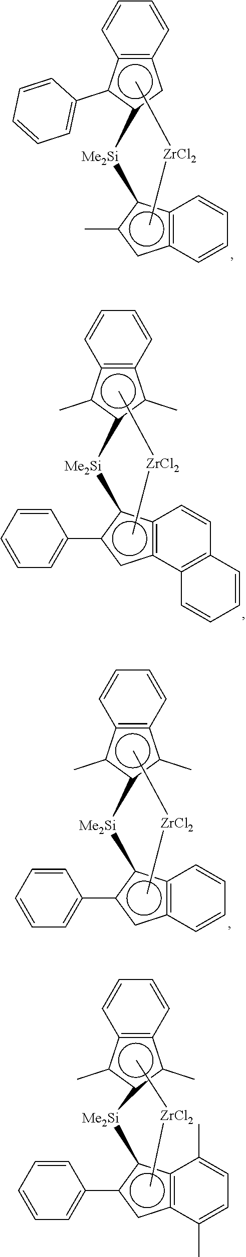

and any combinations thereof.

In various embodiments of the invention, the organic ligand ‘L’ comprises a di-substituted alkyl-silyl bridging group, wherein the bridging group is bonded to two substituted or unsubstituted indenyl groups, wherein at least one substituted or an unsubstituted indenyl group is bonded to the bridging group at the second position of the indenyl group, such as wherein the organometallic complex is:

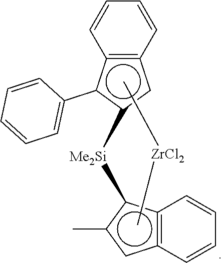

In various embodiments of the invention, the organic ligand ‘L’ comprises a di-substituted alkyl-silyl bridging group, wherein the bridging group is bonded to two substituted or unsubstituted indenyl groups, wherein at least one substituted or an unsubstituted indenyl group is bonded to the bridging group at the second position of the indenyl group, such as wherein the organometallic complex is:

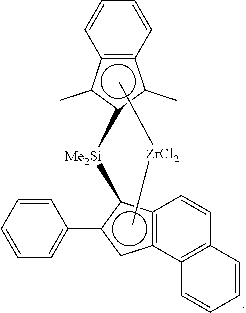

In various embodiments of the invention, the organic ligand ‘L’ comprises a disubstituted alkyl-silyl bridging group, wherein the bridging group is bonded to two substituted or unsubstituted indenyl groups, wherein at least one substituted or an unsubstituted indenyl group is bonded to the bridging group at the second position of the indenyl group, such as wherein the organometallic complex is:

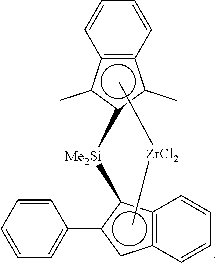

In various embodiments of the invention, the organic ligand ‘L’ comprises a disubstituted alkyl-silyl bridging group, wherein the bridging group is bonded to two substituted or unsubstituted indenyl groups, wherein at least one substituted or an unsubstituted indenyl group is bonded to the bridging group at the second position of the indenyl group, such as wherein the organometallic complex is:

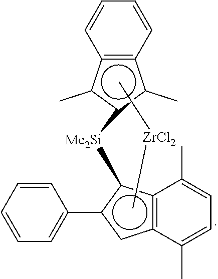

In various embodiments of the invention, the organic ligand ‘L’ comprises a disubstituted alkyl-silyl bridging group, wherein the bridging group is bonded to a tetra-alkyl substituted cyclopentadienyl group and to a 1-indenyl group, wherein the 1-indenyl group comprises a substituent at the second position wherein the substituent is selected from C₃-C₂₀ alkyl or C₆-C₂₀ aryl group, such as wherein the organometallic complex is selected from the group consisting of:

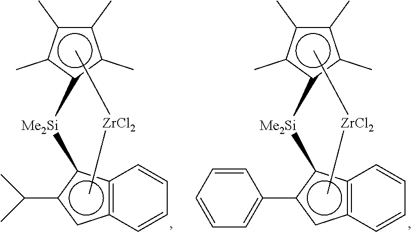

and combinations thereof.

In various embodiments of the invention, the organic ligand ‘L’ comprises a disubstituted alkyl-silyl bridging group, wherein the bridging group is bonded to a tetra-alkyl substituted cyclopentadienyl group and to a 1-indenyl group, wherein the 1-indenyl group comprises a substituent at the second position, such as wherein the substituent is selected from C₃-C₂₀ alkyl or C₆-C₂₀ aryl group, wherein the organometallic complex is

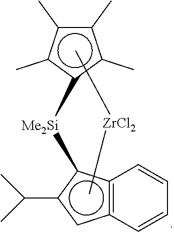

In various embodiments of the invention, the organic ligand ‘L’ comprises a disubstituted alkyl-silyl bridging group, wherein the bridging group is bonded to a tetra-alkyl substituted cyclopentadienyl group and to a 1-indenyl group, wherein the 1-indenyl group comprises a substituent at the second position, such as wherein the substituent is selected from C₃-C₂₀ alkyl or C₆-C₂₀ aryl group, wherein the organometallic complex is

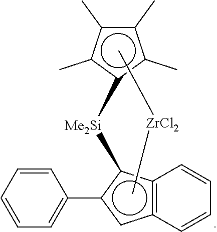

Further examples of organometallic complexes comprising a disubstituted alkyl-silyl bridging group and methods of making such organometallic complexes can be found, for example, in U.S. Pat. No. 11,040,995 to Kulangara et al.; and U.S. Pat. Publ. Nos. US2020/115478A1 to Friederichs et al.; US2020/199165A1 to Friederichs et al.; and US2021/115080A1 to Friederichs et al.; each of which is incorporated by reference in its entirety.

In various embodiments of the invention, the organic ligand ‘L’ comprises a substituted or an unsubstituted 1, 2-phenylene bridging group bonded to a substituted or an unsubstituted 1-indenyl group and to a substituted or an unsubstituted 2-indenyl group; such as wherein the organometallic complex is:

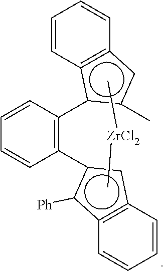

Further examples of organometallic complexes comprising a substituted or an unsubstituted 1, 2-phenylene bridging group and methods of making such organometallic complexes can be found, for example, in U.S. Pat. Publ. No. US2021/079032A1 to Hendriksen et al., which is incorporated by reference in its entirety.

In various embodiments of the invention, the organic ligand ‘L’ comprises a bridging group comprising a substituted or an unsubstituted styryl moiety bonded to a substituent selected from a Group 16 element of the IUPAC Periodic Table and to a substituted cyclopentadienyl group, wherein the bridging group is bonded to a substituted cyclopentadienyl group at the alpha position of the styryl moiety; such as wherein the organometallic complex is selected from the group consisting of:

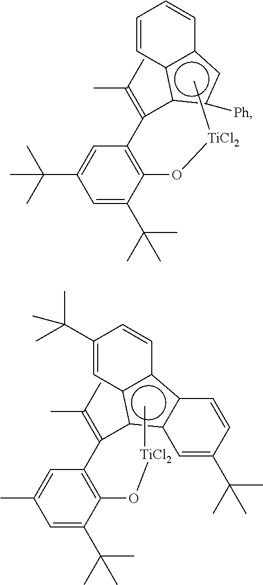

and any combinations thereof.

In various embodiments of the invention, the organic ligand ‘L’ comprises a bridging group comprising a substituted or an unsubstituted styryl moiety bonded to a substituent selected from a Group 16 element of the IUPAC Periodic Table and to a substituted cyclopentadienyl group, wherein the bridging group is bonded to a substituted cyclopentadienyl group at the alpha position of the styryl moiety; such as wherein the organometallic complex is

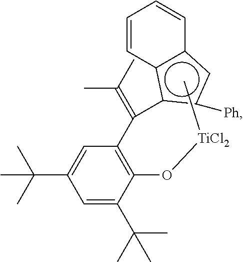

In various embodiments of the invention, the organic ligand ‘L’ comprises a bridging group comprising a substituted or an unsubstituted styryl moiety bonded to a substituent selected from a Group 16 element of the IUPAC Periodic Table and to a substituted cyclopentadienyl group, wherein the bridging group is bonded to a substituted cyclopentadienyl group at the alpha position of the styryl moiety; such as wherein the organometallic complex is

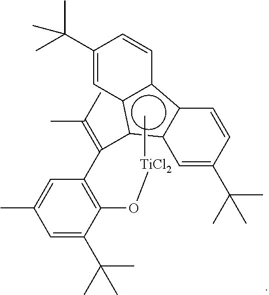

In various embodiments of the invention, the organic ligand ‘L’ comprises a substituted or an unsubstituted 2,2′-biphenylene bridging group bonded to a substituted or an unsubstituted indenyl group, and to a substituent selected from a Group 15 or a Group 16 element of the IUPAC Periodic Table, such as wherein the organometallic complex is:

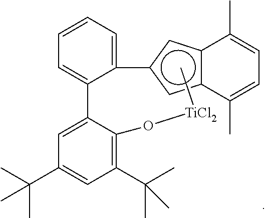

Further examples of organometallic complexes comprising a substituted or an unsubstituted 2,2′-biphenylene bridging group bonded to a substituted or an unsubstituted indenyl group and methods of making such organometallic complexes can be found, for example, in PCT application W02021/048030A1 to Sainani et al., which is incorporated by reference in its entirety.

In various embodiments of the invention, the organic ligand ‘L’ comprises a substituted or an unsubstituted 2,2′-biphenylene group bonded to a substituent (A) and to a substituent (B), wherein substituent (A) is selected from a Group 15 element of the IUPAC Periodic Table and the substituent (B) is selected from a Group 15 or a Group 16 element of the RUPAC Periodic Table, further wherein the group 15 element of substituent (A) is covalently bonded to two sp² hybridized carbon atoms, such as wherein the organometallic complex is:

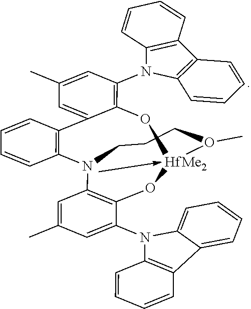

In various embodiments of the invention, the organic ligand ‘L’ comprises a bridging group comprising at least one sp² hybridized carbon atom bonded to two substituted or unsubstituted 2-indenyl groups, such as wherein the organometallic complex is selected from the group consisting of:

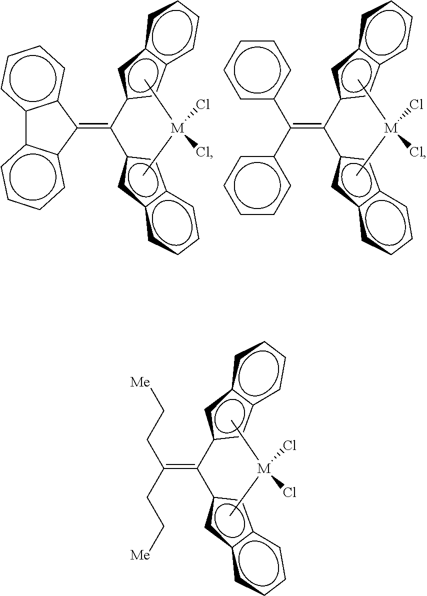

and combinations thereof, wherein ‘M’ is a transition metal having a valency of ‘4’, wherein the metal ‘M’ is selected from Ti, Zr, and Hf.

In various embodiments of the invention, the organic ligand ‘L’ comprises a bridging group comprising at least one sp² hybridized carbon atom bonded to two substituted or unsubstituted 2-indenyl groups, such as wherein the organometallic complex is

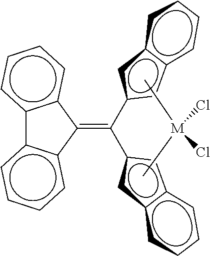

wherein ‘M’ is a transition metal having a valency of ‘4’, wherein the metal ‘M’ is selected from Ti, Zr, and Hf.

In various embodiments of the invention, the organic ligand ‘L’ comprises a bridging group comprising at least one sp² hybridized carbon atom bonded to two substituted or unsubstituted 2-indenyl groups, such as wherein the organometallic complex is

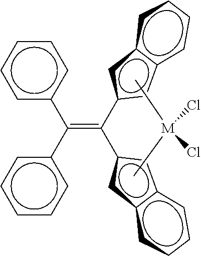

wherein ‘M’ is a transition metal having a valency of ‘4’, wherein the metal ‘M’ is selected from Ti, Zr, and Hf.

In various embodiments of the invention, the organic ligand ‘L’ comprises a bridging group comprising at least one sp² hybridized carbon atom bonded to two substituted or unsubstituted 2-indenyl groups, such as wherein the organometallic complex is

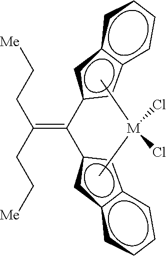

wherein ‘M’ is a transition metal having a valency of ‘4’, wherein the metal ‘M’ is selected from Ti, Zr, and Hf.

In various embodiments of the invention, the organic ligand ‘L’ comprises a substituted or an unsubstituted 1,8-naphthalene-bridging group bonded to two substituted or unsubstituted 2-indenyl groups, such as wherein the organometallic complex is:

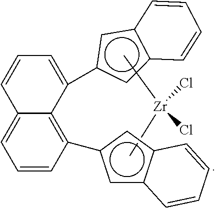

In various preferred embodiments of the invention, the invention relates to a process for producing polyalpha-olefins by polymerizing one or more alpha-olefin monomers in presence of a catalyst composition comprising a reaction product of an organometallic complex and a cocatalyst. Preferably, the cocatalyst is a compound suitable of generating a cationic specie from the organic ligand ‘L’ to form a non- or weakly coordinating anion.

In various embodiments of the invention, the cocatalyst is selected from an aluminium containing cocatalyst, a boron-containing cocatalyst, a zinc containing cocatalyst, or a combination thereof. Suitable aluminium-containing cocatalysts comprise aluminoxanes, alkyl aluminium compounds and aluminium-alkyl-chlorides. The aluminoxanes usable according to the present invention are well known and preferably comprise oligomeric linear and/or cyclic alkyl aluminoxanes represented by the formula: R³—(AlR³—O)ₙ—AlR³₂ for oligomeric, linear aluminoxanes and (—AlR³—O—)ₘ for oligomeric, cyclic aluminoxanes; wherein n is 1-40, preferably n is 10-30; m is 3-40, preferably m is 3-30 and R³ is a C₁ to C₈ alkyl group and preferably a methyl group.

Some alternative examples of alumnimum containing cocatalyst include organoaluminum compounds include trimethylaluminum, triethylaluminium, triisopropylaluminum, tri-n-propylaluminum, triisobutylaluminum, tri-n-butylaluminum, triamylaluminium; dimethylaluminium ethoxide, diethylaluminium ethoxide, diisopropylaluminium ethoxide, di-n-propylaluminium ethoxide, diisobutylaluminium ethoxide and di-n-butylaluminium ethoxide; dimethylaluminium hydride, diethylaluminium hydride, diisopropylaluminium hydride, di-n-propylaluminium hydride, diisobutylaluminium hydride and di-n-butylaluminium hydride, tris-perfluorophenylaluminum.

Suitable boron-containing cocatalysts include trialkylboranes, for example trimethylborane or triethylborane and/or perfluoroarylborane and/or perfluoroarylborate-compounds, triphenylboron, tris-perfluorophenylboron, tetrakisperfluorophenylborate, triphenylcarboniumtetrakis perfluorophenylborate. Suitable zinc containing cocatalyst include diethyl zinc.

In various embodiments of the invention, the catalyst composition further comprises an activator, an anti-static agent, a scavenger. The term “catalyst activator” as used herein is to be understood as any compound, which can activate a single-site catalyst so that it is capable of oligomerizing the alpha-olefin monomers present in the feed stream. Preferably the catalyst activator is an alumoxane, a perfluorophenylborane and/or a perfluorophenylborate, preferably alumoxane, more preferably methylaluminoxane and/or modified methylaluminoxane. Activators that may be used include Lewis acid activators such as triphenylboron, tris-perfluorophenylboron, tris-perfluorophenylaluminum and the like and or ionic activators such as dimethylanilinium tetrakisperfluorophenylborate, triphenylcarboniumtetrakis perfluorophenylborate, dimethyl anilinium tetrakis perfluoro phenyl aluminate, and the like.

In various embodiments of the invention, the catalyst composition may further comprise a co-activator. A co-activator is a compound capable of alkylating the transition metal complex, such that when used in combination with an activator, an active catalyst is formed. Co-activators include alumoxanes such as methylalumoxane, modified alumoxanes such as modified methylalumoxane, and aluminum alkyls such trimethylaluminum, triisobutylaluminum, triethylaluminum, and tri-isopropylaluminum, tri-n-hexylaluminum, tri-n-octylaluminum, tri-n-decylaluminum or tri-n-dodecylaluminum. Co-activators are typically used in combination with Lewis acid activators and ionic activators when the pre-catalyst is not a dihydrocarbyl or dihydride complex. In various embodiments of the invention, co-activators are also used as scavengers to deactivate impurities in feed or reactors. U.S. Pat. No. 9,409,834 B2 (line 39, column 21 to line 44, column 26) provides a detailed description of the activators and coactivators that may be used with a single site catalyst composition. The relevant portions of this patent are incorporated herein by reference in their entirety.

A scavenger is a compound that is typically added to facilitate oligomerization or polymerization by scavenging impurities. Some scavengers may also act as activators and may be referred to as co-activators. A co-activator which is not a scavenger may also be used in conjunction with an activator in order to form an active catalyst with a transition metal compound. In some embodiments, a co-activator can be pre-mixed with the transition metal compound to form an alkylated transition metal compound, also referred to as an alkylated catalyst compound or alkylated metallocene. To the extent scavengers facilitate the single site catalyst composition in performing the intended catalytic function, scavengers, if used, are sometimes considered as a part of the catalyst system.

U.S. Pat. No. 9,409,834 B2, line 37, column 33 to line 61, column 34 provides detailed description of scavengers useful in the process of the present invention for making PAO. The relevant portions in this patent on scavengers, their identities, quantity, and manner of use are incorporated herein in their entirety.

In various embodiments of the invention, the catalyst composition further comprises a support. For example, the support can be an organic or inorganic material and is preferably porous. Examples of organic material are cross-linked or functionalized polystyrene, PVC, cross-linked polyethylene. Examples of inorganic material are silica, alumina, silica-alumina, inorganic chlorides such as magnesium chloride, talc and zeolite. Mixtures of two or more of these supports may be used. The preferred particle size of the support is from 1 to 120 micrometres, preferably of from 20 to 80 micrometres and the preferred average particle size is from 40 to 50 micrometres.

In various embodiments of the invention, the process according to the present invention comprises co-feeding a mixture of alpha-olefin monomers along with the catalyst composition. The reaction may be carried out in batch, semi-batch or continuous, in a single or in multi-stage reactors. In a preferred embodiment, the mixture of catalyst composition and alpha olefin monomers are preferably fed into a first oligomerization reactor, where it is partially reacted and then into a second oligomerization reactor where the reaction may be allowed to continue to completion or where the reaction may be allowed to proceed further and then a subsequent mixture of a catalyst composition, alpha olefin monomers and oligomers are fed into a third oligomerization reactor, where the reaction is completed. Additional oligomerization reactors may be used in series.

It is preferred that each of the reactors may be equipped with a mixing or stirring means for mixing the feed and catalyst to provide intimate contact. In a more preferred embodiment, continuous stirred tank reactors (CSTRs) are used in series. The operation of CSTRs are per se known in the art. Also in an embodiment of the invention, no recycle of unconverted monomer is used. In various embodiments of the present invention, recycle of unconverted monomer is enabled.

Preferably the reaction conditions are controlled so as to cause effective conversion of the alpha-olefin monomers to the desired polyalpha-olefin products. In a preferred embodiment of the present invention, the reactor temperatures are retained between 25° C. and 150° C., preferably between 25° C. and 80° C. and residence time is regulated depending on the reactor type in sequence. For example, the residence time is about 1.5 to about 3 hours in reactor one and about 0.5 to about 1.5 hours in reactor 2, if used. The residence time in a third reactor, if used, would typically be from about 10 minutes to about 1 hour. The reaction is not particularly pressure-dependent and it is most economical to operate the reactors at a low pressure. In various embodiments of the invention, the one or more alpha-olefin monomer is oligomerized at a reactor pressure ranging between atmospheric pressure to about 50 psia.

Optionally, in various embodiments of the invention, a suitable amount of hydrogen may be introduced into the reactor in order to moderate the properties of the polyalpha-olefin so obtained. For example, in a further embodiment of the present invention, the polyalpha-olefin may be hydrogenated by a reaction with hydrogen gas in the presence of a catalytic amount (0.1 to 5 wt. %) of a hydrogenation catalyst. Details pertaining to hydrogenation may be similar to what has been described in the PCT application WO2021086926A1, incorporated herein as reference.

The alpha-olefin monomers may comprise an even number or an odd number of carbon atoms, preferably the alpha-olefin monomer has an even number of carbon atoms. In various embodiments of the invention, polyalpha olefins include monomers having 4-30 carbon atoms, preferably 6 to 18 carbon atoms, and most preferably 8 to 12 carbon atoms. In various embodiments of the invention, the one or more alpha-olefin monomer present in the feed stream is selected from the group consisting of 1-butene, 1-pentene, 1-hexene, 1-heptene, 1-octene, 1-nonene, 1-decene, 1-undecene, 1-dodecene, 1-tridecene, 1-tetradecene, 1-hexadecene, 1-octadecene, 1-eicosene, 4-methyl-1-pentene, 5-methyl-1-nonene, 3-methyl-1-pentene, 3,5,5-trimethyl-1-hexene, vinylcyclohexene, and any combinations thereof. In various embodiments of the invention, the one or more alpha-olefin monomer is oligomerized at a reactor temperature ranging between 25° C. to 150° C.

In various embodiments of the invention, the polyalpha-olefins produced by the present inventive process, comprises C₂₀₋₂₄ dimers, C₃₀₋₃₆ trimers, C₄₀₋₄₈ tetramers, C₅₀₋₆₀ pentamers, and C₆₀₊ heavies. The poly-alpha olefins produced from the process of the present invention may be used for preparing lubricant oil. For example, low viscosity crankcase lubricants consist of a mixture of C₃₀ to C₆₀ hydrocarbons with an average molecular weight (Mw) between 400-850. High viscosity lubricants have a higher Mw as high as 32K (32200), i.e., the equivalent of an oligomer consisting of 230 monomer decene units.

It should be understood that the embodiments and the aspects disclosed herein are not mutually exclusive and such aspects and embodiments can be combined in any way. Those of ordinary skill in the art will readily recognize parameters that can be changed or modified to yield essentially the same results.

What is claimed is:

1. A process for preparing polyalpha-olefins, comprising:
a. providing a feedstream comprising one or more alpha-olefin monomer, wherein the alpha-olefin monomer comprises four to thirty (4-30) carbon atoms;
b. contacting the feedstream with a catalyst composition comprising a reaction product of an organometallic complex and a co-catalyst, wherein the organometallic complex is represented by the general formula:
LMXₙ
wherein:
I. ‘L’ is an organic ligand;
II. ‘M’ is a transition metal having a valency of ‘p’, wherein the metal ‘M’ is selected from Ti, Zr, and Hf;
III. ‘X’ is an anionic ligand to the metal ‘M’, and wherein ‘X’ is selected from the group consisting of halogens, alkyls, aralkyls, alkoxides, amides, and combinations thereof;
IV. ‘n’ is the number of ‘X’ groups and equals p-2;
wherein the organic ligand ‘L’ is selected from the group consisting of:
i. a bridging group bonded to two hydrocarbyl groups, each comprising a substituted or an unsubstituted cyclopentadienyl group; wherein the bridging group contains at least one sp² hybridized carbon atom bonded to at least one of the hydrocarbyl groups;
ii. a substituted or an unsubstituted 2,2′-biphenylene-bridging group, wherein the bridging group is bonded to two hydrocarbyl groups, each comprising a substituted or an unsubstituted cyclopentadienyl group;
iii. a di-substituted alkyl-silyl bridging group, wherein the bridging group is bonded to two substituted or unsubstituted indenyl groups, wherein at least one substituted or unsubstituted indenyl group is bonded to the bridging group at the second position of the indenyl group;
iv. a di-substituted alkyl-silyl bridging group, wherein the bridging group is bonded to a tetra-alkyl substituted cyclopentadienyl group and to a 1-indenyl group, wherein the 1-indenyl group comprises a substituent at the second position wherein the substituent is selected from C₃-C₂₀ alkyl or C₆-C₂₀ aryl group;
v. a substituted or an unsubstituted 1, 2-phenylene bridging group bonded to a substituted or an unsubstituted 1-indenyl group and to a substituted or an unsubstituted 2-indenyl group;
vi. a substituted or an unsubstituted 2,2′-biphenylene-bridging group bonded to two hydrocarbyl groups, each hydrocarbyl group comprising a substituted indenyl group, wherein the bridging group is bonded at the second position of each of the substituted indenyl group;
vii. abridging group comprising a substituted or an unsubstituted styryl moiety bonded to a substituent selected from a Group 16 element of the IUPAC Periodic Table and to a substituted cyclopentadienyl group, wherein the bridging group is bonded to a substituted cyclopentadienyl group at the alpha position of the styryl moiety;
viii. a substituted or an unsubstituted 2,2′-biphenylene bridging group bonded to a substituted or an unsubstituted indenyl group and to a substituent selected from a Group 15 or a Group 16 element of the IUPAC Periodic Table;
ix. a substituted or an unsubstituted 2,2′-biphenylene group bonded to a substituent (A) and to a substituent (B), wherein substituent (A) is selected from a Group 15 element of the IUPAC Periodic Table and substituent (B) is selected from a Group 15 or a Group 16 element of the IUPAC Periodic Table, further wherein the group 15 element of substituent (A) is covalently bonded to two sp² hybridized carbon atoms;
x. a bridging group comprising at least one sp² hybridized carbon atom bonded to two substituted or unsubstituted 2-indenyl groups;
xi. a substituted or an unsubstituted 1,8-naphthalene-bridging group bonded to two substituted or unsubstituted 2-indenyl groups; and
xii. any combination thereof.
c. oligomerizing the one or more alpha-olefin monomer in presence of the catalyst composition under conditions sufficient to produce polyalpha-olefins.

2. The process of claim 1, wherein the organic ligand ‘L’ comprises a substituted or an unsubstituted 2,2′-biphenylene-bridging group, wherein the bridging group is bonded to two hydrocarbyl groups, each comprising a substituted or an unsubstituted cyclopentadienyl group, such as wherein the organometallic complex is selected from the group consisting of: 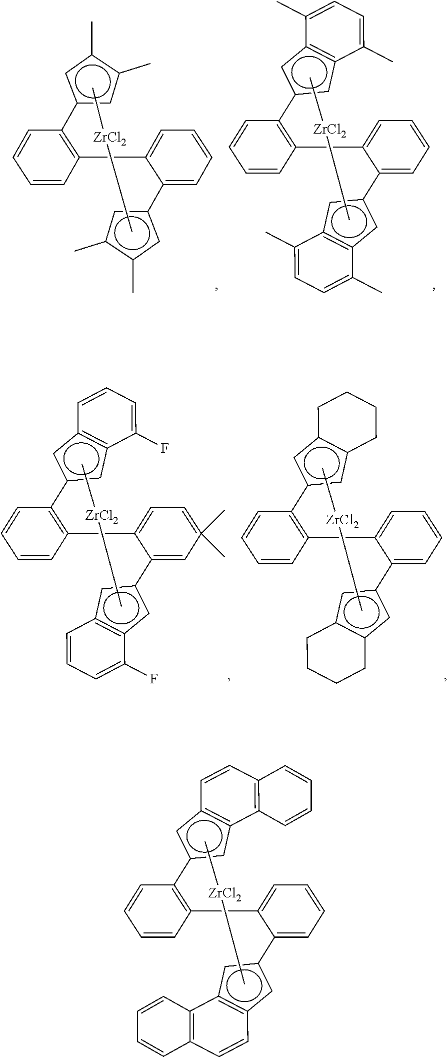
and any combinations thereof.

3. The process of claim 1, wherein the organic ligand ‘L’ comprises a di-substituted alkyl-silyl bridging group, wherein the bridging group is bonded to two substituted or unsubstituted indenyl groups, wherein at least one substituted or an unsubstituted indenyl group is bonded to the bridging group at the second position of the indenyl group, such as wherein the organometallic complex is selected from the group consisting of: 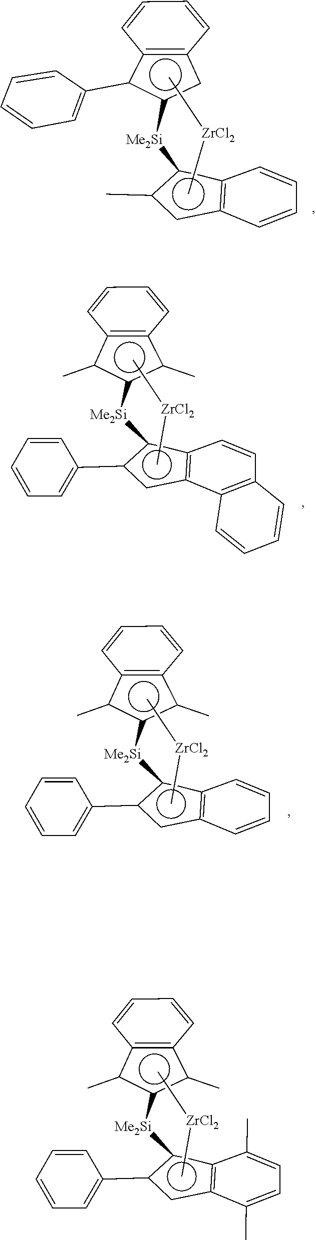
and any combinations thereof.

4. The process of claim 1, wherein the organic ligand ‘L’ comprises a di-substituted alkyl-silyl bridging group, wherein the bridging group is bonded to a tetra-alkyl substituted cyclopentadienyl group and to a 1-indenyl group, wherein the 1-indenyl group comprises a substituent at the second position wherein the substituent is selected from C₃-C₂₀ alkyl or C₆-C₂₀ aryl group, such as wherein the organometallic complex is selected from the group consisting of: 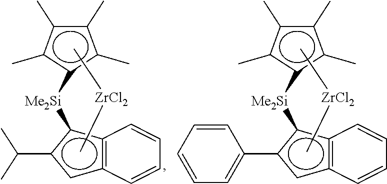
and combinations thereof.

5. The process of claim 1, wherein the organic ligand ‘L’ comprises a substituted or an unsubstituted 1, 2-phenylene bridging group bonded to a substituted or an unsubstituted 1-indenyl group and to a substituted or an unsubstituted 2-indenyl group; such as wherein the organometallic complex is:

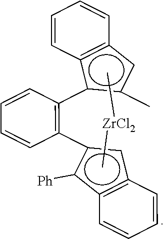

6. The process of claim 1, wherein the organic ligand ‘L’ comprises a substituted or an unsubstituted 2,2′-biphenylene-bridging group bonded to two hydrocarbyl groups, each hydrocarbyl group comprising a substituted indenyl group, wherein the bridging group is bonded at the second position of each of the substituted indenyl group, such as wherein the organometallic complex is:

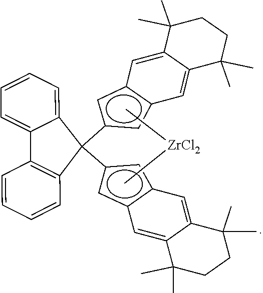

7. The process of claim 1, wherein the organic ligand ‘L’ comprises a bridging group comprising a substituted or an unsubstituted styryl moiety bonded to a substituent selected from a Group 16 element of the IUPAC Periodic Table and to a substituted cyclopentadienyl group, wherein the bridging group is bonded to a substituted cyclopentadienyl group at the alpha position of the styryl moiety; such as wherein the organometallic complex is selected from the group consisting of: 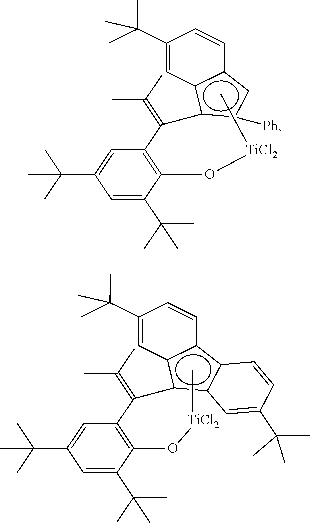
and any combinations thereof.

8. The process of claim 1, wherein the organic ligand ‘L’ comprises a substituted or an unsubstituted 2,2′-biphenylene bridging group bonded to a substituted or an unsubstituted indenyl group and to a substituent selected from a Group 15 or a Group 16 element of the IUPAC Periodic Table, such as wherein the organometallic complex is:

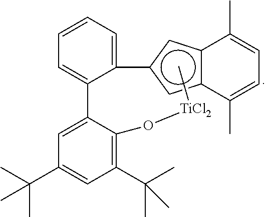

9. The process of claim 1, wherein the organic ligand ‘L’ comprises a substituted or an unsubstituted 2,2′-biphenylene group bonded to a substituent (A) and to a substituent (B), wherein substituent (A) is selected from a Group 15 element of the IUPAC Periodic Table and substituent (B) is selected from a Group 15 or a Group 16 element of the IUPAC Periodic Table, further wherein the group 15 element of substituent (A) is covalently bonded to two sp² hybridized carbon atoms, such as wherein the organometallic complex is:

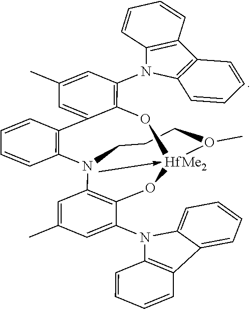

10. The process of claim 1, wherein the organic ligand ‘L’ comprises a bridging group comprising at least one sp² hybridized carbon atom bonded to two substituted or unsubstituted 2-indenyl groups, such as wherein the organometallic complex is selected from the group consisting of: 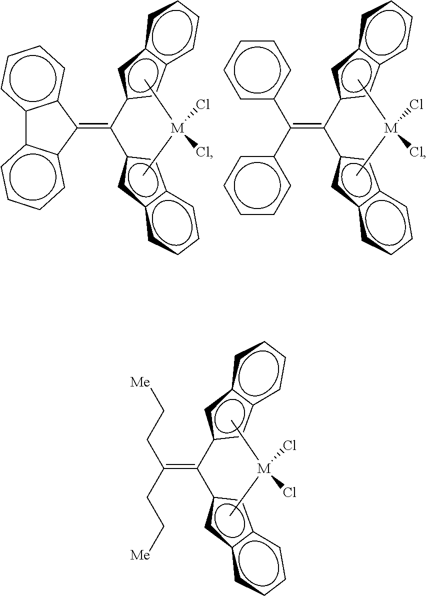
and combinations thereof;
wherein ‘M’ is a transition metal having a valency of ‘4’, wherein the metal ‘M’ is selected from Ti, Zr, and Hf.

11. The process of claim 1, wherein the organic ligand ‘L’ comprises a substituted or an unsubstituted 1,8-naphthalene-bridging group bonded to two substituted or unsubstituted 2-indenyl groups, such as wherein the organometallic complex is:

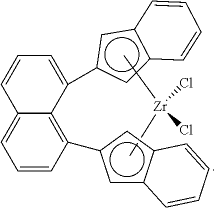

12. The process of claim 1, wherein the one or more alpha-olefin monomer present in the feedstream is selected from the group consisting of 1-butene, 1-pentene, 1-hexene, 1-heptene, 1-octene, 1-nonene, 1-decene, 1-undecene, 1-dodecene, 1-tridecene, 1-tetradecene, 1-hexadecene, 1-octadecene, 1-eicosene, 4-methyl-1-pentene, 5-methyl-1-nonene, 3-methyl-1-pentene, 3,5,5-trimethyl-1-hexene, vinylcyclohexene, and any combinations thereof.

13. The process of claim 1, wherein the one or more alpha-olefin monomer is oligomerized at a reactor temperature ranging between 25° C. to 150° C.

14. The process of claim 1, wherein the one or more alpha-olefin monomer is oligomerized at a reactor pressure ranging between atmospheric pressure to about 50 psia.

15. The process of claim 1, wherein the cocatalyst is selected from an aluminium containing cocatalyst, a boron-containing cocatalyst, zinc containing cocatalyst, or a combination thereof.
